(19) United States
(12) Patent Application Publication
(10) Pub. No.: US 2024/0191264 A1
(43) Pub. Date: Jun. 13, 2024

(54) METHOD FOR PRODUCING HIGHER LINEAR ALKANES

(71) Applicant: Evonik Operations GmbH, Essen (DE)

(72) Inventors: Thomas HAAS, Münster (DE); Christian RICHTER, Münster (DE)

(73) Assignee: Evonik Operations GmbH, Essen (DE)

(21) Appl. No.: 18/285,196

(22) PCT Filed: Mar. 25, 2022
(86) PCT No.: PCT/EP2022/057981

§ 371 Date: Sep. 29, 2023

(30) Foreign Application Priority Data
Mar. 30, 2021 (EP) 21165784.6

(51) Int. Cl. C12P 7/40; C07C 1/24; C07C 5/03; C07C 29/145; C07C 45/48; C07C 45/80

(52) U.S. Cl. CPC C12P 7/40; C07C 1/24; C07C 5/03; C07C 29/145; C07C 45/48; C07C 45/80; C07C 2523/28; C07C 2529/48

(57) ABSTRACT

The present invention relates to a method of producing higher linear alkanes using a combined biotechnological and chemical method. In particular, the present invention relates to producing linear alkanes comprising 7 to 28 carbon atoms, preferably undecane, via higher alkanones, i.e. linear alkanones comprising 7 to 28 carbon atoms, preferably 6-undecanone.

The present invention relates to a method of producing higher linear alkanes using a combined biotechnological and chemical method. In particular, the present invention relates to producing linear alkanes comprising 7 to 28 carbon atoms, preferably undecane, via higher alkanones, i.e. linear alkanones comprising 7 to 28 carbon atoms, preferably 6-undecanone.

Today's fuels need to be replaced or improved in order to reduce carbon dioxide emissions. Both, the national and international regulations/laws require significant savings in carbon dioxide from the transport sector. To meet these requirements, also the existing vehicles (trucks, busses, jets, etc.) must reduce their carbon dioxide emissions. One solution to the savings could be fuels or components of fuels, e.g. higher linear alkanes, such as undecane, synthesized by using carbon dioxide and renewable hydrogen or lower alkanoic acids, e.g. acetic acid or butanoic acid, made therefrom as feedstock basis. Therefore, there is a need of providing a process for the production of higher alkanes, such as undecane, starting from simple organic compounds, such as ethanol and/or lower linear alkanoic acids, such as acetic acid or butanoic acid. Today most of the ethanol is already produced from carbon dioxide, plant scrubbed out of the air. Plants like sugar cane, corn or plant residues are used to form glucose derivatives from sunlight and carbon dioxide derived from air. Alternatively, ethanol can also be formed from carbon dioxide and hydrogen and carbon monoxide using acetogenic organisms like clostridia.

The need to provide a process is satisfied by a method of producing linear alkanes comprising 7 to 28 carbon atoms, from ethanol and/or linear alkanoic acids comprising 2 to 5 carbon atoms, the method comprising
(a) contacting ethanol and/or a linear alkanoic acid comprising 2 to 5 carbon atoms or any salts thereof with at least one microorganism capable of carrying out two-carbon chain elongation to produce as an intermediate a linear alkanoic acid comprising 4 to 7 carbon atoms and/or a salt thereof and/or an ester thereof;
(b) extracting the intermediate, its salt and/or ester thereof from (a) using at least one extractant, wherein the extractant comprises at least one alkyl-phosphine oxide and optionally at least one alkane comprising at least 12 carbon atoms; or at least one trialkylamine and at least one alkane comprising at least 12 carbon atoms; or a branched higher alcohol like 2 octyl-dodecanol;
(c) contacting the extracted intermediate and/or ester thereof from (b) and optionally a further alkanoic acid comprising 1 to 22 carbon atoms with at least one ketonization catalyst to a linear alkanone comprising 7 to 28 carbon atoms;
(d) contacting the linear alkanone comprising 7 to 28 carbon atoms from step (c) with at least one hydrogenation metal catalyst for catalytic hydrogenation of the linear alkanone comprising 7 to 28 carbon atoms to a corresponding secondary linear alkanol comprising 7 to 28 carbon atoms;
(e) dehydration of the secondary alkanol in the presence of an acidic heterogeneous catalyst to form a corresponding linear alkene comprising 7 to 28 carbon atoms; and
(f) contacting the linear alkene comprising 7 to 28 carbon atoms obtained in step (e) with at least one hydrogenation metal catalyst for catalytic hydrogenation to a corresponding secondary linear alkane comprising 7 to 28 carbon atoms;
   or combining step (e) and (f) into one single step using a catalyst or catalyst mixture providing dehydrating and hydrogenating properties, thus hydrogenolysing the secondary linear alkanol comprising 7 to 28 carbon atoms obtained in step (d) directly to the corresponding linear alkane comprising 7 to 28 carbon atoms.

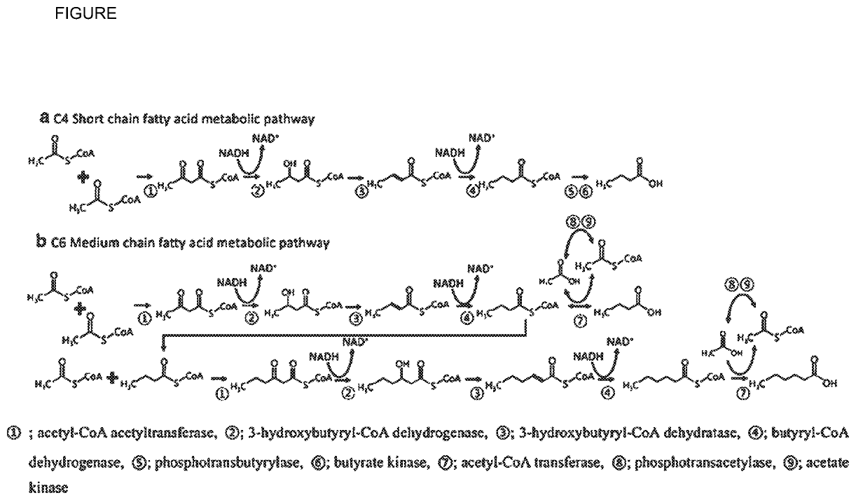

The microorganism in step (a) capable of carrying out carbon chain elongation to produce the intermediate linear alkanoic acid comprising 4 to 7 carbon atoms, e.g. hexanoic acid, may be any organism that may be capable of carbon-chain elongation according to FIG. 1 (Jeon et al. Biotechnol Biofuels (2016) 9:129). The carbon chain elongation pathway is also disclosed in Seedorf, H., et al., 2008. The microorganisms according to any aspect of the present invention may also include microorganisms which in their wild-type form are not capable of carbon chain elongation, but have acquired this trait as a result of genetic modification. In particular, the microorganism in (a) may be selected from the group consisting of Clostridium carboxidivorans and Clostridium kluyveri, more in particular, the microorganism according to any aspect of the present invention may be Clostridium kluyveri.

FIG. 1 shows the microbial metabolic pathway for carbon-chain elongation such as (a) butyric acid (C₄) production by the genera Clostridium and Butyrivibrio (Kim B H, et al. Appl Environ Microbiol. 1984; 48(4):764-70) and (b) hexanoic acid production postulated in Megasphaera elsdenii and Clostridium kluyveri (Khan M A. Melbourne: Victoria University; 2006).

In the method of according to the present invention linear alkanoic acid comprising 2 to 5 carbon atoms may be acetic acid and the intermediate linear alkanoic acid comprising 4 to 7 carbon atoms may be butanoic and hexanoic acid.

In the method of according to the present invention linear alkanoic acid comprising 2 to 5 carbon atoms may be propanoic acid and the intermediate linear alkanoic acid comprising 4 to 7 carbon atoms may be pentanoic acid.

In the method of according to the present invention linear alkanoic acid comprising 2 to 5 carbon atoms may be butanoic acid and the intermediate linear alkanoic acid comprising 4 to 7 carbon atoms may be hexanoic acid.

In the method of according to the present invention linear alkanoic acid comprising 2 to 5 carbon atoms may be pentanoic acid and the intermediate linear alkanoic acid comprising 4 to 7 carbon atoms may be heptanoic acid.

In a preferred embodiment of the method according to the present invention the linear alkanoic acid comprising 2 to 5 carbon atoms is preferably acetic acid and the intermediate linear alkanoic acid comprising 4 to 7 carbon atoms is preferably hexanoic acid or an ester thereof the intermediate alkanoic acid in steps (a) to (c) is preferably hexanoic acid, the linear alkanone comprising 7 to 28 carbon atoms in steps (c) and (d) is preferably 6-undecanone, the corresponding secondary linear alkanol in steps (d) and (e) and in combined steps (e) and (f) is preferably 6-undecanol, the linear alkene in steps (e) and (f) is preferably 5-undecene and the linear alkane is preferably dodecane.

The extraction step in (b) according to any aspect of the present invention allows for an increase in yield relative to the amount of extractants used. For example, less than 50% by weight of extractant may be used to extract the same amount of hexanoic acid as if only pure alkanes were used. Therefore, with a small volume of extractant, a larger yield of intermediate linear alkanoic acid comprising 4 to 7 carbon atoms, e.g. hexanoic acid, may be extracted. The extractant is also not harmful to microorganisms. Accordingly, the extractant according to any aspect of the present invention may be present when the intermediate linear alkanoic acid, e.g. hexanoic acid, is biotechnologically produced according to any aspect of the present invention. Therefore, the aqueous medium according to any aspect of the present invention, particularly after step (b) of separating the intermediate linear alkanoic acid comprising 4 to 7 carbon atoms, e.g. hexanoic acid, may be recycled back into step (a). This step of recycling allows for the microorganisms to be recycled and reused as the extractant according to any aspect of the present invention is not toxic to the microorganisms. This step of recycling the aqueous medium in the method according to any aspect of the present invention has the further advantage of enabling the residue of the intermediate linear alkanoic acid comprising 4 to 7 carbon atoms, e.g. hexanoic acid, which was not at first instance extracted from step (b) in the first cycle, to be given a chance to be extracted a further time or as many times as the aqueous medium is recycled. Further, the intermediate linear alkanoic acid comprising 4 to 7 carbon atoms, e.g. the hexanoic acid, can be easily separated from the extractant according to any aspect of the present invention by distillation. This is because the intermediate linear alkanoic acid comprising 4 to 7 carbon atoms, e.g. hexanoic acid, at least distills at a significantly lower boiling point than the extractant and after the separation via distillation, the extractant may be easily recycled.

The method according to the present invention may also include a step of extracting isolated intermediate linear alkanoic acid comprising 4 to 7 carbon atoms, e.g. hexanoic acid, from an aqueous medium. An isolated intermediate linear alkanoic acid comprising 4 to 7 carbon atoms, e.g. hexanoic acid, may refer to an intermediate linear alkanoic acid, e.g. hexanoic acid, that may be separated from the medium where the intermediate linear alkanoic acid, e.g. hexanoic acid, has been produced. In one example, the intermediate linear alkanoic acid, e.g. hexanoic acid, may be produced in an aqueous medium (e.g. fermentation medium where the intermediate linear alkanoic acid, e.g. hexanoic acid, is produced by specific cells from a carbon source). The isolated intermediate linear alkanoic acid, e.g. hexanoic acid, may refer to the hexanoic acid extracted from the aqueous medium. In particular, the extracting step allows for the separation of excess water from the aqueous medium thus resulting in a formation of a mixture containing the extracted intermediate linear alkanoic acid comprising 4 to 7 carbon atoms, e.g. hexanoic acid.

The extractant may also be referred to as the ‘extraction medium’ or ‘extracting medium’. The extractant may be used for extracting/ isolating the intermediate linear alkanoic acid comprising 4 to 7 carbon atoms, e.g. hexanoic acid, produced according to any method of the present invention from the aqueous medium wherein the intermediate linear alkanoic acid comprising 4 to 7 carbon atoms, e.g. hexanoic acid, was originally produced. At the end of the extracted step, excess water from the aqueous medium may be removed thus resulting in the extractant containing the extracted intermediate linear alkanoic acid comprising 4 to 7 carbon atoms, e.g. hexanoic acid. In particular, at the end of the extracted step, with the intermediate linear alkanoic acid comprising 4 to 7 carbon atoms, e.g. hexanoic acid, extracted and removed, what remains may be the fermentation medium with the cells used for producing the intermediate linear alkanoic acid comprising 4 to 7 carbon atoms, e.g. hexanoic acid, and these cells together with the fermentation medium may then be recycled for step (a). A skilled person would be able to determine if there needs to be a replenishment of the fermentation medium and/or cells after the first cycle. In particular, a first cycle according to any aspect of the present invention involves one round of steps (a) to (c). The medium and/or cells may then be recycled from the second cycle onwards. The extractant may comprise a combination of compounds that may result in an efficient means of extracting the hexanoic acid from the aqueous medium. In particular, the extractant may comprise
at least one alkyl-phosphine oxide and at least one alkane comprising at least 12 carbon atoms; or
at least one trialkylamine and at least one alkane comprising at least 12 carbon atoms.

The extractant according to any aspect of the present invention may efficiently extract the intermediate linear alkanoic acid comprising 4 to 7 carbon atoms, e.g. hexanoic acid, into the extractant. This extractant of a mixture of alkyl-phosphine oxide or trialkylamine and at least one alkane may be considered suitable in the method according to any aspect of the present invention as the mixture works efficiently in extracting the desired intermediate linear alkanoic acid comprising 4 to 7 carbon atoms, e.g. hexanoic acid, in the presence of a fermentation medium. In particular, the mixture of alkyl-phosphine oxide or trialkylamine and at least one alkane may be considered to work better than any method currently known in the art for extraction of the intermediate linear alkanoic acid comprising 4 to 7 carbon atoms, e.g. hexanoic acid, as it does not require any special equipment to be carried out and it is relatively easy to perform with a high product yield. Further, the extractant according to any aspect of the present invention is also not toxic the microorganism according to step (a).

The alkane in the extractant may comprise at least 12 carbon atoms. In particular, the alkane may comprise at least 12-18 carbon atoms. In one example, the alkane may be selected from the group consisting of dodecane, tridecane, tetradecane, pentadecane, hexadecane, heptadecane and octadecane. In a further example, the extractant may comprise a mixture of alkanes. The alkane in the extractant may be used as a solvent for alkyl-phosphine oxide and/or trialkylamine. The presence of the alkane in the extractant allows the extraction temperature to be maintained at a low temperature such that the reaction temperature and extraction temperature is between 35° C.-45° C. More in particular, the reaction temperature of step (a) and extraction temperature may be the optimum temperature for the bacteria according to any aspect of the present invention, namely 37-40° C., more particularly about 37° C. Enabling both processes to be carried out at the simultaneously has the added advantage of being able to extract the alkanoic acid during the extraction step and retaining the cells for further reaction to produce more alkanoic acid to be carried out. Both processes of reaction (to produce alkanoic acid) and extraction (of the alkanoic acid) may take place in the same pot. In another example, both processes take place in 2 different pots. The alkanes used according to any aspect of the present invention comprises at least 12 to 18 carbon atoms as this increasing the boiling point of the alkanes compared to alkanes with less than 12 carbon atoms. The high boiling point of the alkanes enable the separation of the target product (alkanoic acid) from the extractant to be carried out by any method known in the art, for example distillation such that extractant can be separated and reused from the alkanoic acid. Distillation may be carried out at a temperature between 150-350° C.

Alkyl-phosphine oxides have a general formula of OPX₃, where X is an alkyl. Suitable alkyl phosphine oxides according to any aspect of the present invention include an alkyl group composed of a linear, branched or cyclic hydrocarbon, the hydrocarbon composed of from 1 to about 100 carbon atoms and from 1 to about 200 hydrogen atoms. In particular, “alkyl” as used in reference to alkyl phosphine oxide according to any aspect of the present invention can refer to a hydrocarbon group having 1 to 20 carbon atoms, frequently between 4 and 15 carbon atoms, or between 6 and 12 carbon atoms, and which can be composed of straight chains, cyclic, branched chains, or mixtures of these. The alkyl phosphine oxide may have from one to three alkyl groups on each phosphorus atom. In one example, the alkyl phosphine oxide has three alkyl groups on P. In some examples, the alkyl group may comprise an oxygen atom in place of one carbon of a C4-C15 or a C6-C12 alkyl group, provided the oxygen atom is not attached to P of the alkyl phosphine oxide.

Typically, the alkyl phosphine oxide is selected from the group consisting of tri-octylphosphine oxide, tri-butylphosphine oxide, octylphosphine oxide and mixtures thereof. Even more in particular, the alkyl phosphine oxide may be tri-octylphosphine oxide (TOPO).

Trialkylamines are organic-chemical compounds derived from ammonia (NH₃), whose three hydrogen atoms are replaced by alkyl radicals. Examples of trialkylamines are dimethylethylamine, methyldiethylamine, triethylamine, dimethyl-n-propylamine, dimethyl-i-propylamine, methyldi-n-propylamine, dimethylbutylamine, trioctylamine and the like. In particular, the trialkylamine used in the extractant according to any aspect of the present invention may not be soluble in water and may be trioctylamine.

In one example, the extractant according to any aspect of the present invention may be a combination of alkyl-phosphine oxide or trialkylamine and at least one alkane. In particular, the alkane may comprise at least 12 carbon atoms. More in particular, the alkane may comprise at 12-18 carbon atoms. In one example, the alkane may be selected from the group consisting of dodecane, tridecane, tetradecane, pentadecane, hexadecane, heptadecane and octadecane. In a further example, alkane may be a mixture of alkanes.

In a further example, the extractant may comprise a mixture of alkanes. Even more in particular, the extractant according to any aspect of the present invention may be a combination of TOPO and tetradecane or hexadecane. In one example, the extractant according to any aspect of the present invention may be a combination of TOPO and a mixture of alkanes comprising 12-18 carbon atoms.

Trioctylphosphine oxide (TOPO) is an organophosphorus compound with the formula OP(C₈H₁₇)₃. TOPO may be part of the extractant together with at least one alkane according to any aspect of the present invention. In particular, the mixture of TOPO and alkane comprising at least 12 carbon atoms may comprise about 1:100 to 1:10 weight ratio of TOPO relative to the alkane. More in particular, the weight ratio of TOPO to alkane in the extractant according to any aspect of the present invention may be about 1:100, 1:90, 1:80, 1:70, 1:60, 1:50, 1:40, 1:30, 1:25, 1:20, 1:15, or 1:10. Even more in particular, the weight ratio of TOPO to alkane may be selected within the range of 1:90 to 1:10, 1:80 to 1:10, 1:70 to 1:10, 1:60 to 1:10, 1:50 to 1:10, 1:40 to 1:10, 1:30 to 1:10 or 1:20 to 1:10. The weight ratio of TOPO to alkane may be between 1:40 to 1:15 or 1:25 to 1:15. In one example, the weight ratio of TOPO to alkane may be about 1:15. In the example, the alkane may be hexadecane and therefore the weight ratio of TOPO to hexadecane may be about 1:15.

In another example, when the extractant comprises an alkyl-phosphine oxide or a trialkylamine that is more soluble in the alkane used in the extractant compared to the solubility of TOPO in alkane comprising at least 12 carbon atoms, the weight ratio of the alkyl-phosphine oxide (other than TOPO) or a trialkylamine to alkane may be 1:1, 2:1, 3:1, 4:1, 5:1, 6:1, 7:1, 8:1, 9:1 or 10:1. In one example, the extractant may be trihexy-phosphine oxide and the ratio of trihexy-phosphine oxide to alkane may be 1:1. In other examples, the extractant may be a lower chain alkyl-phosphine oxide and the ratio of the lower chain alkyl-phosphine oxide to alkane may be 2:1, 3:1, 4:1, 5:1, 6:1, 7:1, 8:1, 9:1 or 10:1. In this case, a lower-chain alkyl-phosphine oxide refers to a phosphine oxide with a C1-C4 alkyl group. In another example, the extractant may be a trialkylamine, this is known to be more soluble than phosphine oxide in alkanes. For example, the trialkylamine may be a trioctylamine (TOA) that may be present in the extractant according to any aspect of the present invention in the ratio of up to 1:1 with the alkane. Lower chain length amines can be used in even higher ratios. In other examples, the extractant may be a lower chain trialkylamine and the ratio of the lower chain trialkylamine to alkane may be 2:1, 3:1, 4:1, 5:1, 6:1, 7:1, 8:1, 9:1 or 10:1. In this case, a lower-chain alkyl-phosphine oxide refers to a phosphine oxide with a C1-C4 alkyl group.

The term ‘about’ as used herein refers to a variation within 20 percent. In particular, the term “about” as used herein refers to +/−20%, more in particular, +/−10%, even more in particular, +/−5% of a given measurement or value.

Preferably, in step (b) of the method according to the present invention, the alkyl-phosphine oxide is selected from the group consisting of trioctylphosphine oxide, octylphosphine oxide and mixtures thereof, preferably trioctylphosphine oxide (TOPO) and the alkane is selected from the group consisting of pentadecane, hexadecane, heptadecane, octadecane, and tetradecane, preferably tetradecane, preferably in a weight ratio of TOPO to tetradecane between 1:100 to 1:10.

Preferably, the pH of the aqueous medium in step (b) of the method according to the present invention is maintained between 5.5 and 8.

In step (a) according to any aspect of the present invention, ethanol and/or a linear alkanoic acids comprising 2 to 5 carbon atoms, e.g. acetic acid, propanoic acid, butanoic acid or pentanoic acid, or any salts thereof is contacted with at least one microorganism capable of carrying out two-carbon chain elongation to produce the intermediate linear alkanoic acid comprising 4 to 7 carbon atoms, e.g. hexanoic acid, butanoic acid or pentanoic acid, and/or a salt thereof and/or an ester thereof.

In one example, the carbon source may be ethanol in combination with at least one other carbon source selected from the group consisting of acetate, propanoate, butanoate (butyrate) and pentanoate. In particular, the carbon source may be ethanol and acetate. In another example, the carbon source may be a combination of ethanol and butyric (butanoic) acid. In one example, the carbon substrate may be ethanol alone. In another example, the carbon substrate may be the linear alkanoic acids comprising 2 to 5 carbon atoms, e.g. acetate, alone.

The source of the linear alkanoic acids comprising 2 to 5 carbon atoms, e.g. acetate, and/or ethanol may vary depending on availability. In one example, the ethanol and/or the linear alkanoic acids comprising 2 to 5 carbon atoms (e.g. acetate) may be the product of fermentation of synthesis gas (syngas) or any carbohydrate known in the art. In particular, the carbon source for the linear alkanoic acids comprising 2 to 5 carbon atoms (e.g. acetate) and/or ethanol production may be selected from the group consisting of alcohols, aldehydes, glucose, sucrose, fructose, dextrose, lactose, xylose, pentose, polyol, hexose, ethanol and synthesis gas. Mixtures of sources can be used as a carbon source.

Even more in particular, the carbon source may be synthesis gas (syngas). The synthesis gas may be converted to ethanol and/or the linear alkanoic acids comprising 2 to 5 carbon atoms, e.g. acetate, in the presence of at least one acetogenic bacteria.

With respect to the source of substrates comprising carbon dioxide and/or carbon monoxide, a skilled person would understand that many possible sources for the provision of CO and/or CO₂ as a carbon source exist. The syngas or sources of syngas may be derived for instance from steam reforming, partial oxidation or electrochemical synthesis from water or CO₂. It can be seen that in practice, as the carbon source of the present invention any gas or any gas mixture can be used which is able to supply the microorganisms with sufficient amounts of carbon, so that the linear alkanoic acids comprising 2 to 5 carbon atoms (e.g. acetate) and/or ethanol, may be formed from the source of CO and/or CO₂.

Generally, for the acetogenic cell of the present invention the carbon source comprises at least 50% by weight, at least 70% by weight, particularly at least 90% by weight of CO₂ and/or CO, wherein the percentages by weight-% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

The carbon source for the production of the linear alkanoic acids comprising 2 to 5 carbon atoms (e.g. acetate) and/or ethanol used in step (a) according to any aspect of the present invention may be synthesis gas. Synthesis gas can for example be produced as a by-product of coal gasification. Accordingly, the microorganism may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount.

Usually, a portion of the synthesis gas obtained from the gasification process is first processed in order to optimize product yields, and to avoid formation of tar. Cracking of the undesired tar and CO in the synthesis gas may be carried out using lime and/or dolomite.

The overall efficiency, ethanol and/or acetate productivity and/or overall carbon capture may be dependent on the stoichiometry of the CO₂, CO, and H₂ in the continuous gas flow. The continuous gas flows applied may be of composition CO₂ and H₂. In particular, in the continuous gas flow, concentration range of CO₂ may be about 10-50%, in particular 3% by weight and H₂ would be within 44% to 84%, in particular, 64 to 66.04% by weight. In another example, the continuous gas flow can also comprise inert gases like N₂, up to a N₂ concentration of 50% by weight.

More in particular, the carbon source comprising CO and/or CO₂ contacts the acetogenic cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

A skilled person would understand that it may be necessary to monitor the composition and flow rates of the streams at relevant intervals. Control of the composition of the stream can be achieved by varying the proportions of the constituent streams to achieve a target or desirable composition. The composition and flow rate of the blended stream can be monitored by any means known in the art. In one example, the system is adapted to continuously monitor the flow rates and compositions of at least two streams and combine them to produce a single blended substrate stream in a continuous gas flow of optimal composition and means for passing the optimized substrate stream to the fermenter.

A reducing agent, for example hydrogen may be supplied together with the carbon source. In particular, this hydrogen may be supplied when the CO and/or CO₂ is supplied and/or used. In one example, the hydrogen gas is part of the synthesis gas present. In another example, where the hydrogen gas in the synthesis gas is insufficient, additional hydrogen gas may be supplied.

The term “acetogenic bacteria” as used herein refers to a microorganism which is able to perform the Wood-Ljungdahl pathway and thus is able to convert CO, CO₂ and/or hydrogen to a linear alkanoic acids comprising 2 to 5 carbon atoms, e.g. acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui).

More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ERI2, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989.

The production of the intermediate linear alkanoic acid comprising 4 to 7 carbon atoms, its salt and/or ester thereof, e.g. hexanoic acid, is from the linear alkanoic acids comprising 2 to 5 carbon atoms (e.g. acetate) and/or ethanol which is from synthesis gas and may involve the use of the acetogenic bacteria in conjunction with a microorganism capable of carbon chain elongation. For example, Clostridium ljungdahlii may be used simultaneously with Clostridium kluyveri. In another example, a single acetogenic cell may be capable of the activity of both organisms. For example, the acetogenic bacteria may be C. carboxidivorans which may be capable of carrying out both the Wood-Ljungdahl pathway and the carbon chain elongation pathway.

For example, ethanol and/or acetate used in step (a) according to any aspect of the present invention may be a product of fermentation of synthesis gas or may be obtained through other means. The ethanol and/or acetate may then be brought into contact with the microorganism in step (a).

The term “contacting”, as used herein, means bringing about direct contact between the microorganism and the ethanol and/or the linear alkanoic acids comprising 2 to 5 carbon atoms, e.g. acetate. In one example, ethanol is the carbon source and the contacting in step (a) involves contacting the ethanol with the microorganism of step (a). The contact may be a direct contact or an indirect one that may include a membrane or the like separating the cells from the ethanol or where the cells and the ethanol may be kept in two different compartments etc. For example, in step (b) the intermediate linear alkanoic acid comprising 4 to 7 carbon atoms, its salt and/or ester thereof, e.g. the hexanoic acid, and the extracting medium may be in different compartments.

The microorganisms capable of producing the intermediate linear alkanoic acid comprising 4 to 7 carbon atoms, its salt and/or ester thereof, e.g. the hexanoic acid, according to any aspect of the present invention may be cultivated with any culture media, substrates, conditions, and processes generally known in the art for culturing bacteria. This allows for the intermediate linear alkanoic acid comprising 4 to 7 carbon atoms, its salt and/or ester thereof (e.g. hexanoic acid) to be produced using a biotechnological method. Depending on the microorganism that is used for the production of the intermediate linear alkanoic acid comprising 4 to 7 carbon atoms, its salt and/or ester thereof (e.g. hexanoic acid), appropriate growth medium, pH, temperature, agitation rate, inoculum level, and/or aerobic, microaerobic, or anaerobic conditions are varied. A skilled person would understand the other conditions necessary to carry out the method according to any aspect of the present invention. In particular, the conditions in the container (e.g. fermenter) may be varied depending on the microorganisms used. The varying of the conditions to be suitable for the optimal functioning of the microorganisms is within the knowledge of a skilled person.

In one example, the method, in particular step (a) according to any aspect of the present invention may be carried out in an aqueous medium with a pH between 5 and 8, 5.5 and 8 or 5.5 and 7. The pressure may be between 1 and 10 bar. The microorganisms may be cultured at a temperature ranging from about 20° C. to about 80° C. In one example, the microorganism may be cultured at 37° C.

In some examples, for the growth of the microorganism and for its production of the intermediate linear alkanoic acid comprising 4 to 7 carbon atoms, its salt and/or ester thereof (e.g. hexanoic acid), the aqueous medium may comprise any nutrients, ingredients, and/or supplements suitable for growing the microorganism or for promoting the production of the intermediate linear alkanoic acid comprising 4 to 7 carbon atoms, its salt and/or ester thereof, e.g. the hexanoic acid. In particular, the aqueous medium may comprise at least one of the following: carbon sources, nitrogen sources, such as an ammonium salt, yeast extract, or peptone; minerals; salts; cofactors; buffering agents; vitamins; and any other components and/or extracts that may promote the growth of the bacteria. The culture medium to be used must be suitable for the requirements of the particular strains. Descriptions of culture media for various microorganisms are given in “Manual of Methods for General Bacteriology”.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep and/or culture the cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product. For example, for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell. The aqueous medium according to any aspect of the present invention also includes the medium in which the hexanoic acid is produced. It mainly refers to a medium where the solution comprises substantially water. In one example, the aqueous medium in which the cells are used to produce the hexanoic acid is the very medium which contacts the extractant for extraction of the hexanoic acid.

According to any aspect of the present invention it is preferred that the extraction is carried out in step (b) while fermentation takes place in step (a) simultaneously. This depicts an in situ extracting the intermediate linear alkanoic acid comprising 4 to 7 carbon atoms, its salt and/or ester thereof, e.g. the hexanoic acid and/or ester thereof, in step (b).

In step (b) according to any aspect of the present invention, the intermediate linear alkanoic acid comprising 4 to 7 carbon atoms, its salt and/or ester thereof (e.g. the hexanoic acid) in the aqueous medium may contact the extractant for a time sufficient to extract the hexanoic acid from the aqueous medium into the extractant. A skilled person may be capable of determining the amount of time needed to reach distribution equilibrium and the right bubble agglomeration that may be needed to optimize the extraction process. In some examples the time needed may be dependent on the amount of hexanoic acid that may be extracted. In particular, the time needed to extract the intermediate linear alkanoic acid comprising 4 to 7 carbon atoms, its salt and/or ester thereof (e.g. the hexanoic acid) from the aqueous medium into the extractant may only take a few minutes. According to any aspect of the present invention, where the extraction is carried out in step (b) as fermentation takes place in step (a), the time for extraction may be equivalent to the time of fermentation.

The ratio of the extractant used to the amount of hexanoic acid to be extracted may vary depending on how quick the extraction is to be carried out. In one example, the amount of extractant is equal to the amount of aqueous medium comprising the intermediate linear alkanoic acid comprising 4 to 7 carbon atoms, its salt and/or ester thereof, e.g. the hexanoic acid. After the step of contacting the extractant with the aqueous medium, the two phases (aqueous and organic) are separated using any means known in the art. In one example, the two phases may be separated using a separation funnel. The two phases may also be separated using mixer-settlers, pulsed columns, and the like. In one example, the separation of the extracting medium from the intermediate linear alkanoic acid comprising 4 to 7 carbon atoms, its salt and/or ester thereof, e.g. the hexanoic acid, may be carried out using distillation in view of the fact that the intermediate linear alkanoic acid comprising 4 to 7 carbon atoms, e.g. hexanoic acid, distills at a significantly lower boiling point than the extractant. A skilled person may be able to select the best method of separating the extractant from the desired hexanoic acid in step (b) depending on the characteristics of the intermediate linear alkanoic acid comprising 4 to 7 carbon atoms, its salt and/or ester thereof, e.g. hexanoic acid. In particular, step (c) according to any aspect of the present invention involves the recovering of the intermediate linear alkanoic acid comprising 4 to 7 carbon atoms, its salt and/or ester thereof, e.g. the hexanoic acid, from step (b). The intermediate linear alkanoic acid comprising 4 to 7 carbon atoms, its salt and/or ester thereof, e.g. hexanoic acid, brought into contact with the organic extractant results in the formation of two phases, the two phases (aqueous and organic) are separated using any means known in the art. In one example, the two phases may be separated using a separation funnel. The two phases may also be separated using mixer-settlers, pulsed columns, thermal separation and the like. In one example, where the intermediate linear alkanoic acid comprising 4 to 7 carbon atoms is hexanoic acid, the separation of the extracting medium from the hexanoic acid may be carried out using distillation in view of the fact that hexanoic acid distills at a significantly lower boiling point than the extracting medium.

Step (b) ends with the organic absorbent made available again to be recycled or reused.

Accordingly, the method of extraction of hexanoic acid according to any aspect of the present invention may be used together with any biotechnological method of producing the hexanoic acid. This is especially advantageous as usually during the fermentation process to produce hexanoic acid using biological methods, the hexanoic acid would be left to collect in the aqueous medium and after reaching certain concentrations in the fermentation medium, the very target product (hexanoic acid) may inhibit the activity and productivity of the microorganism. This thus limits the overall yield of the fermentation process. With the use of this extraction method, the intermediate linear alkanoic acid comprising 4 to 7 carbon atoms, its salt and/or ester thereof, e.g. the hexanoic acid, is extracted as it is produced thus reducing end-product inhibition drastically.

The method according to any aspect of the present invention is also more efficient and cost-effective than the traditional methods of removing hexanoic acid, particularly from a fermentation method as it is produced, as there is no primary reliance on distillation and/or a precipitation for recovering of hexanoic acids. Distillation or precipitation process may lead to higher manufacturing costs, lower yield, and higher waste products therefore reducing the overall efficiency of the process. The method according to any aspect of the present invention attempts to overcome these shortcomings.

In particular, the mixture of the microorganism and the carbon source according to any aspect of the present invention may be employed in any known bioreactor or fermenter to carry out any aspect of the present invention. In one example, the complete method according to any aspect of the present invention that begins with the biotechnological production of the intermediate linear alkanoic acid comprising 4 to 7 carbon atoms, its salt and/or ester thereof, e.g. the hexanoic acid from acetate and/or ethanol and ends with the extraction of the intermediate linear alkanoic acid comprising 4 to 7 carbon atoms, its salt and/or ester thereof, e.g. hexanoic acid, takes place in a single container. There may therefore be no separation step between the step of producing the intermediate linear alkanoic acid comprising 4 to 7 carbon atoms, its salt and/or ester thereof, e.g. hexanoic acid, and the step of extracting the intermediate linear alkanoic acid comprising 4 to 7 carbon atoms, its salt and/or ester thereof, e.g. the hexanoic acid. This saves time and costs. In particular, during the fermentation process, the microorganism may be grown in the aqueous medium and in the presence of the extractant. The method according to any aspect of the present invention thus provides for a one pot means of producing the intermediate linear alkanoic acid comprising 4 to 7 carbon atoms, its salt and/or ester thereof, e.g. of the hexanoic acid. Also, since the intermediate linear alkanoic acid comprising 4 to 7 carbon atoms, its salt and/or ester thereof, e.g. the hexanoic acid, is being extracted as it is produced, no end-product inhibition takes place, ensuring that the yield of hexanoic acid is maintained. A further step of separation may be carried out to remove the intermediate linear alkanoic acid comprising 4 to 7 carbon atoms, its salt and/or ester thereof, e.g. the hexanoic acid. Any separation method known in the art such as using a funnel, column, distillation and the like may be used. The remaining extractant and/or the cells may then be recycled.

In another example, the extraction process of the intermediate linear alkanoic acid comprising 4 to 7 carbon atoms, its salt and/or ester thereof, e.g. the hexanoic acid, may take place as a separate step and/or in another pot. After fermentation has taken place, where the desired intermediate linear alkanoic acid comprising 4 to 7 carbon atoms, e.g. hexanoic acid, to be extracted has already been produced, the extractant according to any aspect of the present invention may be added to the fermentation medium or the fermentation medium may be added to a pot comprising the extractant. The desired intermediate linear alkanoic acid comprising 4 to 7 carbon atoms, e.g. hexanoic acid, may then be extracted by any separation method known in the art such as using a funnel, column, distillation and the like. The remaining extractant may then be recycled. The fermentation medium with the cells may also be recycled.

Another advantage of the method is that the extractant may be recycled. Therefore, once the intermediate linear alkanoic acid comprising 4 to 7 carbon atoms, e.g. hexanoic acid, is separated from extractant, the extractant can be recycled and reused, reducing waste.

Step (c) of the method according to any aspect of the present invention involves contacting the extracted the intermediate linear alkanoic acid comprising 4 to 7 carbon atoms, its salt and/or ester thereof, e.g. the hexanoic acid, from (b) with at least one ketonization catalyst and optionally a further alkanoic acid comprising 1 to 22 carbon atoms under suitable reaction conditions for chemical ketonization of the intermediate linear alkanoic acid comprising 4 to 7 carbon atoms, its salt and/or ester thereof, e.g. the hexanoic acid, e.g. hexanoic acid, to linear alkanones comprising 7 to 28 carbon atoms, preferably 6-undecanone.

The term “further alkanoic acid comprising 1 to 22 carbon atoms” does not encompass hexanoic acid. Preferably the further alkanoic acid comprising 1 to 22 carbon atoms is selected from straight chain alkanoic acids comprising 4 to 18, preferably 5 to 12, carbon atoms.

A ketonization catalyst according to any aspect of the present invention may be any metal oxide catalyst or mixtures thereof. Ketonization reacts the intermediate linear alkanoic acid comprising 4 to 7 carbon atoms, its salt and/or ester thereof, e.g. the hexanoic acid, with the further alkanoic acid and/or the intermediate linear alkanoic acid comprising 4 to 7 carbon atoms, its salt and/or ester thereof, e.g. the hexanoic acid, preferably dimerizes two molecules of the intermediate linear alkanoic acid comprising 4 to 7 carbon atoms (e.g. hexanoic acid), to one ketone molecule with the removal of one water and one carbon dioxide. In case of hexanoic acid being the intermediate linear alkanoic acid comprising 4 to 7 carbon atoms, the mechanism that may be involved in ketonization of the intermediate linear alkanoic acid comprising 4 to 7 carbon atoms where hexanoic anhydride ((CH₃(CH₂)₄)COOCO(CH₂)₄CH₃) may be formed is disclosed at least in Woo, Y., Ind. Eng. Chem. Res. 2017, 56: 872-880. Ketonization of hexanoic acid in the presence of a variety of metal oxide catalysts is at also shown in Wang, S. J. Phys. Chem. C 2017, 121, 18030-18046.

The ketonization catalyst used according to any aspect of this present invention may be a heterogeneous catalyst for the efficient production of a higher-energy-density ketone, preferably C11, from biologically produced hexanoic acid according to step (a). In particular, a ketonization catalyst may be any metal oxide catalyst or mixtures thereof selected from the group consisting of metal oxide catalyst or mixtures thereof is selected from the group consisting of heteropoly acid (H₃PW₁₂O₄₀) catalyst, niobium oxide (Nb₂O₅) catalyst, titanium oxide (TiO₂) catalyst, cerium oxide (CeO₂) catalyst, zinc-chromium (Zn—Cr) mixed oxide catalyst, manganese oxide (MnOₓ) catalyst, lanthanum oxide (La₂O₃) catalyst, magnesium oxide (MgO) catalyst, iron oxide (FeO, FeO₂, Fe₂O₃, Fe₃O₄, Fe₄O₅, Fe₅O₆, Fe₅O₇), silicon-aluminium (Si_{y}Al_{z}O) mixed oxide catalyst, aluminium oxide (Al₂O₃) catalyst and zirconia (ZrO₂) catalyst. The ‘x’ in MnOₓ may be 1, 2 or 4. The ‘y’ and ‘z’ in Si_{y}Al_{z}O may refer to any number where the ratio z/y is any number between 0 to 1. In one example, ketonization is carried out on hexanoic acid as disclosed in Pham T. N., ACS Catal. 2013, 3: 2456-2473 using a suitable heterogenous hydrogenation metal catalyst and suitable reaction conditions. The conditions as disclosed can vary depending on the catalyst used for effective yield of linear alkanones comprising 7 to 28 carbon atoms, e.g. 6-undecanone. In yet another example, MnO₂ and/or Al₂O₃ catalyst may be used based on what is disclosed in Gliński, M. et al, Polish J. Chem. 2004, 78: 299-302 for ketonizing hexanoic acid to 6-undecanone. In a further example, Nb₂O₅ catalyst may be used as disclosed in U.S. Pat. No. 6,265,618 B1 especially in example 3 in ketonizing hexanoic acid to 6-undecanone. A skilled person would by simple trial and error be able to identify the suitable catalyst and the appropriate conditions for producing linear alkanones comprising 7 to 28 carbon atoms, preferably 6-undecanone, from hexanoic acid and eventually another alkanoic acid based on the state of the art. Orozco, L. M et al ChemSusChem, 2016, 9(17): 2430-2442 and Orozco, L. M et al Green Chemistry, 2017, 19(6): 1555-1569 also disclose other catalyst that may be used as ketonization catalysts according to any aspect of the present invention.

The metal oxide catalyst or mixtures thereof is preferably selected from the group consisting of heteropoly acid (H₃PW₁₂O₄₀) catalyst, titanium oxide (TiO₂) catalyst, cerium oxide (CeO₂) catalyst, zinc-chromium (Zn—Cr) mixed oxide catalyst, manganese oxide (MnO₂) catalyst, lanthanum oxide (La₂O₃) catalyst, magnesium oxide (MgO) catalyst, iron oxide (FeO, FeO₂, Fe₂O₃, Fe₃O₄, Fe₄O₅, Fe₅O₆, Fe₅O₇), silicon-aluminium (Si—Al) mixed oxide catalyst and zirconia (ZrO₂) catalyst. Preferably, the ketonization catalyst in step (c) is a manganese oxide (MnOₓ) catalyst, magnesium oxide (MgO) catalyst, or zirconia aerogel catalyst. In particular, the ketonization catalyst in step (c) may be a manganese oxide (MnOₓ) catalyst, or a magnesium oxide (MgO) catalyst.

It would be within the knowledge of a skilled person to determine the suitable conditions for the use of the different ketonization catalysts in step (c). In particular, the ketonization catalyst may be a zirconia aerogel catalyst and may be used in the ketonization of hexanoic acid as disclosed in Woo, Y., Ind. Eng. Chem. Res. 2017, 56: 872-880. The zirconia aerogel catalyst may not only efficiently produce linear alkanones comprising 7 to 28 carbon atoms, preferably 6-undecanone, but it also avoids leaching of the catalysts. Lee, Y. et al in Applied Catalysis A: General. 2015, 506: 288-293 discloses different ketonization catalysts and their effectiveness in ketonization hexanoic acid. A skilled person can very easily use the method described in Lee Y., et al to determine the suitable ketonization catalysts and/or conditions for use in the ketonization of the intermediate linear alkanoic acid comprising 4 to 7 carbon atoms, e.g. hexanoic acid.

In particular, suitable reaction conditions of step (c) comprises reaction temperatures of 100° C.-500° C., 100° C.-450° C., 100° C.-400° C., 100° C.-350° C., 100° C.-300° C., 100° C.-250° C., 100° C.-200° C., 150° C.-500° C., 150° C.-450° C., 150° C.-400° C., 150° C.-350° C., 150° C. -300° C., 150° C.-250° C., 150° C.-200° C., 200° C.-500° C., 200° C.-450° C., 200° C.-400° C., 200° C.-350° C., 200° C.-300° C., 200° C.-250° C., 250° C.-500° C., 250° C.-450° C., 250° C.-400° C., 250° C.-350° C., 250° C.-300° C. and the like.

Preferably, the suitable reaction conditions of step (c) comprises reaction temperatures of 150° C.-350° C.

More in particular, linear alkanones comprising 7 to 28 carbon atoms, preferably 6-undecanone, may be produced from hexanoic acid using MgO/SiO₂ catalyst as the ketonization catalyst at reaction temperatures of 150° C.-350° C., preferably at reaction temperatures of 200° C.-350° C.

Step (d) of the method according to the present invention provides contacting the linear alkanone comprising 7 to 28 carbon atoms, preferably 6-undecanone, obtained in step (c) with at least one hydrogenation metal catalyst for catalytic hydrogenation of the linear alkanone comprising 7 to 28 carbon atoms, preferably 6-undecanone, to a corresponding secondary linear alkanol comprising 7 to 28 carbon atoms, preferably 6-undecanol.

In particular step (d) of the method according to the present invention comprises contacting the linear alkanone comprising 7 to 28 carbon atoms, preferably the 6-undecanone, produced according to any aspect of the present invention with at least one hydrogenation metal catalyst for catalytic hydrogenation of the linear alkanone comprising 7 to 28 carbon atoms, preferably 6-undecanone, to secondary linear alkanol comprising 7 to 28 carbon atoms, preferably 6-undecanol. Higher alkanols, preferably 6-undecanol (C₁₁H₂₄O), a secondary alcohol, are a result of the catalytic hydrogenation of the linear alkanone comprising 7 to 28 carbon atoms, preferably 6-undecanone, a molecule of hydrogen is added across the carbon-oxygen double bond to ultimately furnish the higher alkanol, preferably 6-undecanol, as the final product.

The hydrogenation metal catalyst may be a homogeneous or heterogeneous catalyst.

Homogeneous metal catalysts may be metal complexes that are known in the art. In particular, the hydrogenation metal catalyst may be a heterogeneous catalyst. Some advantages of using multiphase catalytic reactions using solid catalysts include easy separation of catalysts and products, easy recovery, and catalyst recycling, and relatively mild operating conditions. There are also clear economic and environmental incentives in using heterogeneous catalysts. In particular, the hydrogenation metal catalyst may be selected from the group consisting of ruthenium (Ru) catalyst, rhenium (Re) catalyst, nickel (Ni) catalyst, iron (Fe), cobalt (Co), palladium (Pd) catalyst and platinum (Pt) catalyst. More in particular, the catalyst may be selected from the group consisting of Ni, Pd and Pt catalyst. In one example, the hydrogenation metal catalyst used according to any aspect of the present invention may be nickel nanoparticles as described in Alonso, F. Tetrahedron, 2008, 64: 1847-52. In another example, Iron(II) PNP Pincer Complexes may be used as the hydrogenation metal catalyst for hydrogenation of the linear alkanone comprising 7 to 28 carbon atoms to the higher secondary alcohol, preferably from 6-undecanone to 6-undecanol, as disclosed in Gorgas, N., Organometallics, 2014, 33 (23): 6905-6914. In yet another example, magnetite nanoparticles of ruthenium (Ru) catalyst, rhenium (Re) catalyst, nickel (Ni) catalyst, iron (Fe), cobalt (Co), palladium (Pd) catalyst or platinum (Pt) catalyst as described in Tariq Shah M., et al., ACS Applied Materials & Interfaces, 2015: 7(12), 6480-9 may be used as the heterogenous hydrogenation metal catalyst according to any aspect of the present invention.

In yet another example, a copper-phosphine complex is used as a homogeneous hydrogenation metal catalyst according to any aspect of the present invention as disclosed in Chen, J-X., Tetrahedron, 2000, 56: 2153-2166. In a further example, a heterogenous Pt catalyst, in particular a Pt/Al₂O₃ catalyst, as disclosed in Journal of Molecular Catalysis A: Chemical, 2014, 388-389: 116-122 may be used in hydrogenation of the linear alkanone comprising 7 to 28 carbon atoms to the higher secondary alcohol, preferably from 6-undecanone to 6-undecanol. ChemSusChem, 2017: 10(11), 2527-2533 also discloses a variety of heterogenous catalysts such as Pt/C, Ru/C, and Pd/C that may be used in combination with or without an acid catalyst for the hydrogenation of 6-undecanone to 6-undecanol. Based on the above, a skilled person may determine a suitable hydrogenation catalyst to be used according to any aspect of the present invention to yield the higher alkanol from the linear alkanone comprising 7 to 28 carbon atoms, preferably 6-undecanol from 6-undecanone.

A skilled person would easily be able to determine the suitable hydrogenation metal catalyst and vary the conditions accordingly to efficiently produce a secondary linear alkanol comprising 7 to 28 carbon atoms, preferably 6-undecanol, from hydrogenation of 6-undecanone.

The hydrogenation metal catalyst of step (d) of the method according to the present invention is preferably selected from the group consisting of ruthenium (Ru) catalyst, rhenium (Re) catalyst, nickel (Ni) catalyst, iron (Fe), cobalt (Co) and platinum (Pt) catalyst.

Step (e) of the method according to the present invention comprises a dehydration of the secondary linear alkanol comprising 7 to 28 carbon atoms, e.g. 6-undecanol in the presence of an acidic heterogeneous catalyst to form a corresponding higher alkene, e.g. 5-undecene.

Preferably, the acidic heterogeneous catalyst of step (e) is a catalyst comprising an aluminosilicate zeolite. In a particularly preferred embodiment of the method according to the present invention the acidic heterogeneous catalyst of step (e) comprises ZSM-5.

ZSM-5, Zeolite Socony Mobil-5 (framework type MFI from ZSM-5 (five)), is an aluminosilicate zeolite belonging to the pentasil family of zeolites. Its chemical formula is NaₙAlₙSi₉₆₋ₙO₁₉₂·16H₂O (0<n<27). Patented by Mobil Oil Company in 1975, it is widely used in the petroleum industry as a heterogeneous catalyst for hydrocarbon isomerization reactions.

The acidic heterogeneous catalyst of step (e) may further comprise a transition metal, selected from palladium, nickel, cobalt, iron, molybdenum and manganese, preferably molybdenum, preferably 5% molybdenum by weight.

The reaction temperature of the dehydration step (e) is from about 100 to about 250° C., preferably from about 120 to about 180° C. The solvent may be toluene.

Steps (e), dehydration of the secondary linear alkanol comprising 7 to 28 carbon atoms, and (f), the catalytic hydrogenation of the resulting linear alkene comprising 7 to 28 carbon atoms to the corresponding linear alkane comprising 7 to 28 carbon atoms, may be combined in a single step using a catalyst or catalyst mixture providing dehydrating and hydrogenating properties.

### EXAMPLES

### Example 1

### Clostridium kluyveri Forming Hexanoic Acid From Acetate and Ethanol

For the biotransformation of ethanol and acetate to hexanoic acid the bacterium Clostridium kluyveri was used. All cultivation steps were carried out under anaerobic conditions in pressure-resistant glass bottles that can be closed airtight with a butyl rubber stopper.

For the preculture 100 ml of DMSZ52 medium (pH=7.0; 10 g/L K-acetate, 0.31 g/L K₂HPO₄, 0.23 g/L KH₂PO₄, 0.25 g/l NH₄Cl, 0.20 g/l MgSO₄×7 H₂O, 1 g/L yeast extract, 0.50 mg/L resazurin, 10 μl/I HCl (25%, 7.7 M), 1.5 mg/L FeCl₂×4H₂O, 70 μg/L ZnCl₂×7H₂O, 100 μg/L MnCl₂×4H₂O, 6 μg/L H₃BO₃, 190 μg/L CoCl₂×6H₂O, 2 μg/L CuCl₂×6H₂O, 24 μg/L NiCl₂×6H₂O, 36 μg/L Na₂MO₄×2H₂O, 0.5 mg/L NaOH, 3 μg/L Na₂SeO₃×5H₂O, 4 μg/L Na₂WO₄×2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine —HCl×2H₂O, 20 ml/L ethanol, 2.5 g/L NaHCO₃, 0.25 g/L cysteine-HCl×H₂O, 0.25 g/L Na₂S×9H₂O) in a 250 ml bottle were inoculated with 5 ml of a frozen cryoculture of Clostridium kluyveri and incubated at 37° C. for 144 h to an OD₆₀₀ₙₘ>0.2.

For the main culture 200 ml of fresh DMSZ52 medium in a 500 ml bottle were inoculated with centrifuged cells from the preculture to an OD₆₀₀ₙₘ of 0.1. This growing culture was incubated at 37° C. for 27 h to an OD₆₀₀ₙₘ>0.6. Then the cell suspension was centrifuged, washed with production buffer (pH 6.0; 0.832 g/L K-acetate, 5.0 g/l ethanol) and centrifuged again.

For the production culture, 200 ml of production buffer in a 500 ml bottle was inoculated with the washed cells from the main culture to an OD₆₀₀ₙₘ of 0.2. The culture was capped with a butyl rubber stopper and incubated for 71 h at 37° C. and 100 rpm in an open water shaking bath. At the start and end of the culturing period, samples were taken. These were tested for optical density, pH and the different analytes (tested by NMR).

The results showed that in the production phase the amount of acetate decreased from 0.54 g/l to 0.03 g/l and the amount of ethanol decreased from 5.6 g/l to 4.9 g/l. Also, the concentration of butyric acid was increased from 0.05 g/l to 0.28 g/l and the concentration of hexanoic acid was increased from 0.03 g/l to 0.79 g/l.

### Example 2

### Clostridium kluyveri Forming Hexanoic Acid From Butyric Acid and Ethanol

For the biotransformation of ethanol and butyric acid to hexanoic acid the bacterium Clostridium kluyveri was used. All cultivation steps were carried out under anaerobic conditions in pressure-resistant glass bottles that can be closed airtight with a butyl rubber stopper.

For the preculture 100 ml of DMSZ52 medium (pH=7.0; 10 g/L K-acetate, 0.31 g/L K₂HPO₄, 0.23 g/L KH₂PO₄, 0.25 g/l NH₄Cl, 0.20 g/l MgSO₄×7 H₂O, 1 g/L yeast extract, 0.50 mg/L resazurin, 10 μl/l HCl (25%, 7.7 M), 1.5 mg/L FeCl₂×4H₂O, 70 μg/L ZnCl₂×7H₂O, 100 μg/L MnCl₂×4H₂O, 6 μg/L H₃BO₃, 190 μg/L CoCl₂×6H₂O, 2 μg/L CuCl₂×6H₂O, 24 μg/L NiCl₂×6H₂O, 36 μg/L Na₂MO₄×2H₂O, 0.5 mg/L NaOH, 3 μg/L Na₂SeO₃×5H₂O, 4 μg/L Na₂WO₄×2H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine —HCl×2H₂O, 20 ml/L ethanol, 2.5 g/L NaHCO₃, 0.25 g/L cysteine-HCl×H₂O, 0.25 g/L Na₂S×9H₂O) in a 250 ml bottle were inoculated with 5 ml of a frozen cryoculture of Clostridium kluyveri and incubated at 37° C. for 144 h to an OD₆₀₀ₙₘ>0.3.

For the main culture 200 ml of fresh DMSZ52 medium in a 500 ml bottle were inoculated with centrifuged cells from the preculture to an OD₆₀₀ₙₘ of 0.1. This growing culture was incubated at 37° C. for 25 h to an OD₆₀₀ₙₘ>0.4. Then the cell suspension was centrifuged, washed with production buffer (pH 6.16; 4.16 g/L K-acetate, 10.0 g/l ethanol) and centrifuged again.

For the production cultures, 200 ml of production buffer in a 500 ml bottle was inoculated with the washed cells from the main culture to an OD₆₀₀ₙₘ of 0.2. In a first culture, at the beginning 1.0 g/l butyric acid was added to the production buffer, in a second culture, no butyric acid was added to the production buffer. The cultures were capped with a butyl rubber stopper and incubated for 71 h at 37° C. and 100 rpm in an open water shaking bath. At the start and end of the culturing period, samples were taken. These were tested for optical density, pH and the different analytes (tested by NMR).

The results showed that in the production phase of the butyric acid supplemented culture the amount of acetate decreased from 3.1 g/l to 1.1 g/l and the amount of ethanol decreased from 10.6 g/l to 7.5 g/l. Also, the concentration of butyric acid was increased from 1.2 g/l to 2.2 g/l and the concentration of hexanoic acid was increased from 0.04 g/l to 2.30 g/l.

In the production phase of the non-supplemented culture the amount of acetate decreased from 3.0 g/l to 1.3 g/l and the amount of ethanol decreased from 10.2 g/l to 8.2 g/l. Also, the concentration of butyric acid was increased from 0.1 g/l to 1.7 g/l and the concentration of hexanoic acid was increased from 0.01 g/l to 1.40 g/l.

### Example 3

### Cultivation of Clostridium kluyveri in Presence of Decane and TOPO

The bacterium Clostridium kluyveri was cultivated for the biotransformation of ethanol and acetate to hexanoic acid. For the inSitu extraction of the produced hexanoic acid a mixture of decane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄×7 H₂O, 10 μl/L HCl (7.7 M), 1.5 mg/L FeCl₂×4 H₂O, 36 μg/L ZnCl₂, 64 μg/L MnCl₂×4 H₂O, 6 μg/L H₃BO₃, 190 μg/L CoCl₂×6 H₂O, 1.2 μg/L CuCl₂×6 H₂O, 24 μg/L NiCl₂×6 H₂O, 36 μg/L Na₂MO₄×2 H₂O, 0.5 mg/L NaOH, 3 μg/L Na₂SeO₃×5 H₂O, 4 μg/L Na₂WO₄×2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl×2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD₆₀₀ₙₘ of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously fed to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle was inoculated with centrifuged cells from the preculture to an OD₆₀₀ₙₘ of 0.1. Additional 1 ml of a mixture of 6% (w/w) TOPO in decane was added. The culture was capped with a butyl rubber stopper and incubated at 37° C. and 150 rpm in an open water bath shaker for 43 h under 100% CO₂ atmosphere. During cultivation several 5 mL samples were taken to determinate OD₆₀₀ₙₘ, PH und product formation. The determination of the product concentrations was performed by semi-quantitative 1H-NMR spectroscopy. As an internal quantification standard sodium trimethylsilylpropionate (T(M)SP) was used.

During the main cultivation the concentration of butyrate increased from 0.14 g/L to 2.12 g/L and the concentration of hexanoate increased from 0.22 g/L to 0.91 g/L, whereas the concentration of ethanol decreased from 15.04 to 11.98 g/l and the concentration of acetate decreased from 6.01 to 4.23 g/L.

The OD₆₀₀ₙₘ decreased during this time from 0.111 to 0.076.

### Example 4

### Cultivation of Clostridium kluyveri in Presence of Tetradecane and TOPO

The bacterium Clostridium kluyveri was cultivated for the biotransformation of ethanol and acetate to hexanoic acid. For the inSitu extraction of the produced hexanoic acid a mixture of tetradecane with trioctylphosphineoxide (TOPO) was added to the cultivation. All cultivation steps were carried out under anaerobic conditions in pressure-resistant glass bottles that can be closed airtight with a butyl rubber stopper.

The precultivation of Clostridium kluyveri was carried out in a 1000 mL pressure-resistant glass bottle in 250 ml of EvoDM24 medium (pH 5.5; 0.429 g/L Mg-acetate, 0.164 g/l Na-acetate, 0.016 g/L Ca-acetate, 2.454 g/l K-acetate, 0.107 mL/L H₃PO₄ (8.5%), 0.7 g/L NH₄acetate, 0.35 mg/L Co-acetate, 1.245 mg/L Ni-acetate, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 0.702 mg/L (NH4)₂Fe(SO₄)₂×4 H₂O, 1 ml/L KS-acetate (93.5 mM), 20 mL/L ethanol, 0.37 g/L acetic acid) at 37° C., 150 rpm and a ventilation rate of 1 L/h with a mixture of 25% CO₂ and 75% N₂ in an open water bath shake. The gas was discharged into the headspace of the reactor. The pH was hold at 5.5 by automatic addition of 2.5 M NH₃ solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor and hold an OD₆₀₀ₙₘ of ˜1.5.

For the main culture 100 ml of Veri01 medium (pH 6.5; 10 g/L potassium acetate, 0.31 g/L K₂HPO₄, 0.23 g/L KH₂PO₄, 0.25 g/L NH₄Cl, 0.20 g/L MgSO₄×7 H₂O, 10 μl/L HCl (7.7 M), 1.5 mg/L FeCl₂×4 H₂O, 36 μg/L ZnCl₂, 64 μg/L MnCl₂×4 H₂O, 6 μg/L H₃BO₃, 190 μg/L CoCl₂×6 H₂O, 1.2 μg/L CuCl₂×6 H₂O, 24 μg/L NiCl₂×6 H₂O, 36 μg/L Na₂MO₄×2 H₂O, 0.5 mg/L NaOH, 3 μg/L Na₂SeO₃×5 H₂O, 4 g/L Na₂WO₄×2 H₂O, 100 μg/L vitamin B12, 80 μg/L p-aminobenzoic acid, 20 μg/L D(+) Biotin, 200 g/L nicotinic acid, 100 μg/L D-Ca-pantothenate, 300 μg/L pyridoxine hydrochloride, 200 μg/l thiamine-HCl×2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine, 2.5 mL/L HCL 25%) in a 250 ml bottle were inoculated with centrifuged cells from the preculture to an OD₆₀₀ₙₘ of 0.1. Additional 1 ml of a mixture of 6% (w/w) TOPO in tetradecane was added. The culture was capped with a butyl rubber stopper and incubated at 37° C. and 150 rpm in an open water bath shaker for 47 h under 100% CO₂ atmosphere.

During cultivation several 5 mL samples were taken to determinate OD₆₀₀ₙₘ, PH und product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.05 g/L to 3.78 g/L and the concentration of hexanoate increased from 0.09 g/L to 4.93 g/L, whereas the concentration of ethanol decreased from 15.52 to 9.36 g/l and the concentration of acetate decreased from 6.36 to 2.49 g/L.

The OD₆₀₀ₙₘ increased during this time from 0.095 to 0.685.

### Example 5

### Cultivation of Clostridium kluyveri in Presence of Hexadecane and TOPO

The bacterium Clostridium kluyveri was cultivated for the biotransformation of ethanol and acetate to hexanoic acid. For the inSitu extraction of the produced hexanoic acid a mixture of hexadecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄×7 H₂O, 10 μl/L HCl (7.7 M), 1.5 mg/L FeCl₂×4 H₂O, 36 μg/L ZnCl₂, 64 μg/L MnCl₂×4 H₂O, 6 μg/L H₃BO₃, 190 μg/L CoCl₂×6 H₂O, 1.2 μg/L CuCl₂×6 H₂O, 24 μg/L NiCl₂×6 H₂O, 36 μg/L Na₂MO₄×2 H₂O, 0.5 mg/L NaOH, 3 μg/L Na₂SeO₃×5 H₂O, 4 μg/L Na₂WO₄×2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl×2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD₆₀₀ₙₘ of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously fed to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle was inoculated with centrifuged cells from the preculture to an OD₆₀₀ₙₘ of 0.1. Additional 1 ml of a mixture of 6% (w/w) TOPO in hexadecane was added. The culture was capped with a butyl rubber stopper and incubated at 37° C. and 150 rpm in an open water bath shaker for 43 h under 100% CO₂ atmosphere.

During cultivation several 5 mL samples were taken to determinate OD₆₀₀ₙₘ, pH und product formation. The determination of the product concentrations was performed by semi-quantitative 1H-NMR spectroscopy. As an internal quantification standard sodium trimethylsilylpropionate (T(M)SP) was used.

During the main cultivation the concentration of butyrate increased from 0.14 g/L to 2.86 g/L and the concentration of hexanoate increased from 0.20 g/L to 2.37 g/L, whereas the concentration of ethanol decreased from 14.59 to 10.24 g/l and the concentration of acetate decreased from 5.87 to 3.32 g/L.

The OD₆₀₀ₙₘ increased during this time from 0.091 to 0.256.

### Example 6

### Cultivation of Clostridium kluyveri in Presence of Heptadecane and TOPO

The bacterium Clostridium kluyveri was cultivated for the biotransformation of ethanol and acetate to hexanoic acid. For the inSitu extraction of the produced hexanoic acid a mixture of heptadecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄×7 H₂O, 10 μl/L HCl (7.7 M), 1.5 mg/L FeCl₂×4 H₂O, 36 μg/L ZnCl₂, 64 μg/L MnCl₂×4 H₂O, 6 μg/L H₃BO₃, 190 μg/L CoCl₂×6 H₂O, 1.2 μg/L CuCl₂×6 H₂O, 24 μg/L NiCl₂×6 H₂O, 36 μg/L Na₂MO₄×2 H₂O, 0.5 mg/L NaOH, 3 μg/L Na₂SeO₃×5 H₂O, 4 μg/L Na₂WO₄×2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl×2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD₆₀₀ₙₘ of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle were inoculated with centrifuged cells from the preculture to an OD₆₀₀ₙₘ of 0.1. Additional 1 ml of a mixture of 6% (w/w) TOPO in heptadecane was added. The culture was capped with a butyl rubber stopper and incubated at 37° C. and 150 rpm in an open water bath shaker for 43 h under 100% CO₂ atmosphere.

During cultivation several 5 mL samples were taken to determinate OD₆₀₀ₙₘ, PH und product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.15 g/L to 2.82 g/L and the concentration of hexanoate increased from 0.19 g/L to 2.85 g/L, whereas the concentration of ethanol decreased from 14.34 to 9.58 g/l and the concentration of acetate decreased from 5.88 to 3.20 g/L.

The OD₆₀₀ₙₘ increased during this time from 0.083 to 0.363.

### Example 7

### Cultivation of Clostridium kluyveri in Presence of Dodecane and TOPO

The bacterium Clostridium kluyveri was cultivated for the biotransformation of ethanol and acetate to hexanoic acid. For the inSitu extraction of the produced hexanoic acid a mixture of dodecane with trioctylphosphineoxide (TOPO) was added to the cultivation. All cultivation steps were carried out under anaerobic conditions in pressure-resistant glass bottles that can be closed airtight with a butyl rubber stopper.

For the preculture 250 ml of Veri01 medium (pH 7.0; 10 g/L potassium acetate, 0.31 g/L K₂HPO₄, 0.23 g/L KH₂PO₄, 0.25 g/L NH₄Cl, 0.20 g/L MgSO₄×7 H₂O, 10 μl/L HCl (7.7 M), 1.5 mg/L FeCl₂×4 H₂O, 36 μg/L ZnCl₂, 64 μg/L MnCl₂×4 H₂O, 6 μg/L H₃BO₃, 190 μg/L CoCl₂×6 H₂O, 1.2 μg/L CuCl₂×6 H₂O, 24 μg/L NiCl₂×6 H₂O, 36 μg/L Na₂MO₄×2 H₂O, 0.5 mg/L NaOH, 3 μg/L Na₂SeO₃×5 H₂O, 4 μg/L Na₂WO₄×2 H₂O, 100 μg/L vitamin B12, 80 μg/L p-aminobenzoic acid, 20 μg/L D(+) Biotin, 200 μg/L nicotinic acid, 100 μg/L D-Ca-pantothenate, 300 μg/L pyridoxine hydrochloride, 200 μg/l thiamine-HCl×2H₂O, 20 ml/L ethanol, 2.5 g/L NaHCO₃, 65 mg/L glycine, 24 mg/L histidine, 64.6 mg/L isoleucine, 93.8 mg/L leucine, 103 mg/L lysine, 60.4 mg/L arginine, 21.64 mg/L L-cysteine-HCl, 21 mg/L methionine, 52 mg/L proline, 56.8 mg/L serine, 59 mg/L threonine, 75.8 mg/L valine) were inoculated with 10 ml of a living culture of Clostridium kluyveri to a start OD₆₀₀ₙₘ of 0.1.

The cultivation was carried out in a 1000 mL pressure-resistant glass bottle at 37° C., 150 rpm and a ventilation rate of 1 L/h with 100% CO₂ in an open water bath shaker for 671 h. The gas was discharged into the headspace of the reactor. The pH was hold at 6.2 by automatic addition of 100 g/L NaOH solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor.

For the main culture 100 ml of fresh Veri01 medium in a 250 ml bottle were inoculated with centrifuged cells from the preculture to an OD₆₀₀ₙₘ of 0.1. Additional 1 ml of a mixture of 6% (w/w) TOPO in dodecane was added. The culture was capped with a butyl rubber stopper and incubated at 37° C. and 150 rpm in an open water bath shaker for 43 h under 100% CO₂ atmosphere. During cultivation several 5 mL samples were taken to determinate OD₆₀₀ₙₘ, pH und product formation. The determination of the product concentrations was performed by semiquantitative 1H-NMR spectroscopy. As an internal quantification standard sodium trimethylsilylpropionate (T(M)SP) was used.

During the main cultivation the concentration of butyrate increased from 0.14 g/L to 2.62 g/L and the concentration of hexanoate increased from 0.22 g/L to 2.05 g/L, whereas the concentration of ethanol decreased from 14.62 to 10.64 g/l and the concentration of acetate decreased from 5.92 to 3.54 g/L.

The OD₆₀₀ₙₘ increased during this time from 0.091 to 0.259.

### Example 8

### Determination of the Distribution Coefficient for Hexanoic Acid Between Water and a Mixture of Hexadecane and TOPO

During all stages of the experiment, samples from both phases were taken for determination of pH and concentration of hexanoic acid by high performance liquid chromatography (HPLC). 100 g of an aqueous solution of 5 g/kg hexanoic acid and 33 g of a mixture of 6% trioctylphosphinoxide (TOPO) in hexadecane were filled in a separatory funnel and mixed for 1 minute at 37° C. Then the funnel was placed in a tripod ring and the emulsion was left to stand to separate spontaneously. The pH of the aqueous phase was measured. Then 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system of water and 6% TOPO in hexadecane was calculated from the concentrations of hexanoic acid in both phases.

$$K ( D ) = \frac{c ( Hex , organic phase )}{c ( Hex , aqueous phase )}$$

The K_{D} for hexanoic acid in the system of water and 6% TOPO in hexadecane at pH 6.2 was 4.7.

### Example 9

### Determination of the Distribution Coefficient for Hexanoic Acid Between Water and a Mixture of Heptadecane and TOPO

During all stages of the experiment, samples from both phases were taken for determination of pH and concentration of hexanoic acid by high performance liquid chromatography (HPLC). 100 g of an aqueous solution of 5 g/kg hexanoic acid and 33 g of a mixture of 6% trioctylphosphinoxide (TOPO) in heptadecane were filled in a separatory funnel and mixed for 1 minute at 37° C. Then the funnel was placed in a tripod ring and the emulsion was left to stand to separate spontaneously. The pH of the aqueous phase was measured. 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system of water and 6% TOPO in heptadecane was calculated from the concentrations of hexanoic acid in both phases.

$$K ( D ) = \frac{c ( Hex , organic phase )}{c ( Hex , aqueous phase )}$$

The K_{D} for hexanoic acid in the system water and 6% TOPO in heptadecane at pH 6.2 was 5.0.

### Example 10

### Determination of the Distribution Coefficient for Hexanoic Acid Between Water and a Mixture of Tetradecane and TOPO

During all stages of the experiment, samples from both phases were taken for determination of pH and concentration of hexanoic acid by high performance liquid chromatography (HPLC). 130 g of an aqueous solution of 5 g/kg hexanoic acid plus 0.5 g/kg acetic acid and 15 g of a mixture of 6% trioctylphosphinoxid (TOPO) in tetradecane were filled in a separatory funnel and mixed for 1 minute at 37° C. Then the funnel was placed in a tripod ring and the emulsion was led stand to separate spontaneously. The pH of the aqueous phase was measured. 1M NaOH solution was added to the funnel and mixed. The step of separation and sampling was repeated until a pH of 6.2 in the aqueous phase was reached. Samples from both phases were taken for later analysis at this point. The aqueous phase could be analyzed directly by HPLC. For the analysis of the organic phase the diluted hexanoic acid was first re-extracted to water (pH 12.0 by addition of 1 M NaOH) and then analyzed by HPLC. The distribution coefficient K_{D} of hexanoic acid in the system water and 6% TOPO in tetradecane was calculated from the concentrations of hexanoic acid in both phases.

$$K ( D ) = \frac{c ( Hex , organic phase )}{c ( Hex , aqueous phase )}$$

The K_{D} for hexanoic acid in the system water and 6% TOPO in tetradecane at pH 6.9 was 1.3.

### Example 11

### Cultivation of Clostridium kluyveri and Extraction of Hexanoic Acid

The bacterium Clostridium kluyveri was cultivated for the biotransformation of ethanol and acetate to hexanoic acid. For the inSitu extraction of the produced hexanoic acid a mixture of tetradecane with trioctylphosphineoxide (TOPO) was continuously passed through the cultivation. All cultivation steps were carried out under anaerobic conditions in pressure-resistant glass bottles that can be closed airtight with a butyl rubber stopper.

The precultivation of Clostridium kluyveri was carried out in a 1000 mL pressure-resistant glass bottle in 250 ml of EvoDM45 medium (pH 5.5; 0.004 g/L Mg-acetate, 0.164 g/l Na-acetate, 0.016 g/L Ca-acetate, 0.25 g/l K-acetate, 0.107 mL/L H₃PO₄ (8.5%), 2.92 g/L NH₄acetate, 0.35 mg/L Co-acetate, 1.245 mg/L Ni-acetate, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 0.702 mg/L (NH4)₂Fe(SO₄)₂×4 H₂O, 1 ml/L KS-acetate (93.5 mM), 20 mL/L ethanol, 0.37 g/L acetic acid) at 37° C., 150 rpm and a ventilation rate of 1 L/h with a mixture of 25% CO₂ and 75% N₂ in an open water bath shaker. The gas was discharged into the headspace of the reactor. The pH was hold at 5.5 by automatic addition of 2.5 M NH₃ solution. Fresh medium was continuously feeded to the reactor with a dilution rate of 2.0 d⁻¹ and fermentation broth continuously removed from the reactor through a KrosFlo® hollow fibre polyethersulfone membrane with a pore size of 0.2 μm (Spectrumlabs, Rancho Dominguez, USA) to retain the cells in the reactor and hold an OD₆₀₀ₙₘ of ˜1.5.

For the main culture 150 ml of EvoDM39 medium (pH 5.8; 0.429 g/L Mg-acetate, 0.164 g/l Na-acetate, 0.016 g/L Ca-acetate, 2.454 g/l K-acetate, 0.107 mL/L H₃PO₄ (8.5%), 1.01 mL/L acetic acid, 0.35 mg/L Co-acetate, 1.245 mg/L Ni-acetate, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 0.702 mg/L (NH4)₂Fe(SO₄)₂×4 H₂O, 1 ml/L KS-acetate (93.5 mM), 20 mL/L ethanol, 8.8 mL NH₃ solution (2.5 mol/L), 27.75 ml/L acetic acid (144 g/L))

in a 1000 ml bottle were inoculated with 100 ml cell broth from the preculture to an OD₆₀₀ₙₘ of 0.71.

The cultivation was carried out at 37° C., 150 rpm and a ventilation rate of 1 L/h with a mixture of 25% CO₂ and 75% N₂ in an open water bath shaker for 65 h. The gas was discharged into the headspace of the reactor. The pH was hold at 5.8 by automatic addition of 2.5 M NH₃ solution. Fresh medium was continuously feeded to the reactor with a dilution rate of 0.5 d⁻¹ and fermentation broth continuously removed from the reactor by holding an OD₆₀₀ₙₘ of ˜0.5. Additional 120 g of a mixture of 6% (w/w) TOPO in tetradecane was added to the fermentation broth. Then this organic mixture was continuously feeded to the reactor and the organic phase also continuously removed from the reactor with a dilution rate of 1 d⁻¹.

During cultivation several 5 mL samples from both, the aqueous and the organic phase, were taken to determinate OD₆₀₀ₙₘ, pH und product formation. The determination of the product concentrations was performed by semiquantitative 1H-NMR spectroscopy. As an internal quantification standard sodium trimethylsilylpropionate (T(M)SP) was used.

During the main cultivation in the aqueous phase a steady state concentration of 8.18 g/L ethanol, 3.20 g/L acetate, 1.81 g/L butyrate and 0.81 g/L hexanoate was reached. The OD₆₀₀ₙₘ remained stable at 0.5. In the organic phase a steady state concentration of 0.43 g/kg ethanol, 0.08 g/kg acetate, 1.13 g/kg butyrate and 8.09 g/kg hexanoate was reached. After the experiment the cells remained viable while transferred to further cultivations.

The distribution coefficient K_{D} of the substrates and products in the system aqueous medium and 6% TOPO in tetradecane was calculated from the concentrations in both phases.

$$K ( D ) = \frac{c ( organic phase )}{c ( aqueous phase )}$$

The K_{D} in the steady state was 0.05 for ethanol, 0.03 for acetic acid, 0.62 for butyric acid and 9.99 for hexanoic acid.

### Example 12

### Ketonization of Hexanoic Acid

The ketonization was conducted in a heated continuous flow-bed reactor. At first, the reactor was charged with magnesium oxide on silica (50 wt. %, 14.00 g) and heated under an argon flow (54 mL/min) at 330° ° C. for one hour. The temperature was raised to 360° C. Than a mixture of hexanoic acid in tetradecane (v/v: 3/1) was continuously fed to the reactor with a rate of 3.3 mL/h. The gaseous out stream was collected by two cooling traps, which were cooled with water and a mixture of dry ice and isopropanol. The collected fractions were weighted and analyzed by gas chromatography (GC) for their composition. In total, 370.65 g of hexanoic acid was fed to the reactor, which equals to a maximum theoretical yield of 271.70 g of 6-undecanone and 28.75 g of water and 70.21 g of carbon dioxide as by-products. The obtained amount of 6-undecanone was 267.67 g and the amount of water was 28.32 g. This corresponds to a 99% mass recovery at full conversion. The high productivity and selectivity were confirmed by regular GC measurements, as only traces of hexanoic acid and no side-products were detected.

### Example 13

### Cross Ketonization of Hexanoic Acid With Palmitic Acid

The technical procedure of the cross ketonization is identical to the sole ketonization of hexanoic acid (example 12) except the composition of the substrate feed. The feed consists of hexanoic acid (116.16 g, 1.00 mol) and palmitic acid (256.43 g, 1.00 mol) as substrates and tetradecane (124.20 g) as internal standard. The substrate feed is added with a rate of 3.3 mL/h and reacted at a temperature of 360° C. The presence of two alkanoic acids leads two a product mixture of three ketones: 6-undecanone, 6-henicosanone and 16-hentriacontanone. At full conversion, the amounts obtained are 42.58 g of 6-undecanone, 155.29 g of 6-henicosanone and 112.71 g of 16-hentriacontanone.

### Example 14

### Cross Ketonization of Hexanoic Acid With Acetic Acid

The technical procedure of the cross ketonization is identical to the sole ketonization of hexanoic acid (example 12) except the composition of the substrate feed. The feed consists of hexanoic acid 232.32 g, 2.00 mol) and acetic acid (120.10 g, 2.00 mol) as substrates and tetradecane (117.47 g) as internal standard. The substrate feed is added with a rate of 3.3 mL/h and reacted at a temperature of 360° C. The presence of two alkanoic acids leads two a product mixture of three ketones: 2-propanone, 2-heptanone and 6-undecanone. At full conversion, the amounts obtained are 29.04 g of 2-propanone, 114.19 g of 2-heptanone and 85.15 g of 6-undecanone.

### Example 15

### Hydrogenation of the Higher Linear Alkanone to a Corresponding Higher Linear Alkane

The hydrogenation reaction of the C₁₁ ketone (6-undecanone) to the linear alcane (undecane) was performed in a 300 ml autoclave reactor (PARR Instrument Company). The reactor was placed in an aluminum block and the temperature was controlled by a thermocouple placed inside the reactor. Typically, 30 mg of solid catalyst, 170.3 mg, 1.0 mmol of substrate was added to a 4 ml glass vial having an oven dried magnetic stirrer. 2.0 ml of dry Toluene was used as solvent, vial was fitted with a screw cap and a needle was inserted through the septum. (The vial is placed in the reactor.) The reactor was purged three times with 10 bar of H₂ and then the pressure was increased to 20 bar. The reactor was heated to the desired temperature (120° C.) for 20 h. After the reaction, the reactor was cooled down to 5° C. using an ice bath, the gas phase was slowly released and the remaining liquid was carefully separated from the solid catalyst and was analyzed separately using an internal standard (100 μL n-hexadecane).

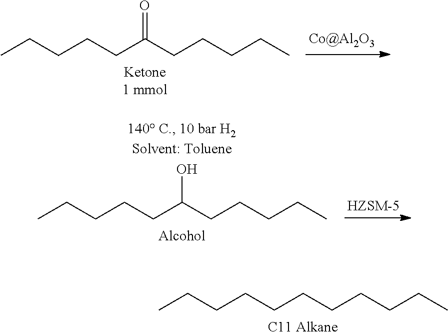

The ketone hydrogenation was investigated on a supported heterogeneous catalyst, 3.0Co@γ-Al₂O₃, and added H-ZSM5 zeolith. This mixture showed 99% ketone conversion and an alcane yield of 98%.

The metal catalyst preparation method is as follows:

3 wt %Co@γ-Al₂O₃, using Ascorbic acid as reductant and glucose as capping agent in H₂O, Pyrolysis at 800° C. for 2 h, Co salt is Cobalt(II) nitrate hexahydrate. In a typical synthesis, 149 mg, 0.51 mmol of Co(NO₃)₂. 6H₂O was dissolved in 20 ml D.I H₂O followed by the stepwise addition of aqueous solutions of 265 mg, 3.0 mmol Ascorbic acid and 92 mg, 1 mmol of D-(+)-Glucose. The contents were stirred at 90° C. for 2-3 h. Next, 1.0 g of γ-Al₂O₃ support was added and the slurry was stirred overnight at R.T. Excess water was removed through centrifugation and the solids were dried in oven at 120° C. for 10 h and then pyrolyzed at 800° C. for 2 h under argon atmosphere.

1. A method of producing linear alkanes comprising 7 to 28 carbon atoms, from ethanol and/or linear alkanoic acids comprising 2 to 5 carbon atoms, the method comprising
(a) contacting ethanol and/or a linear alkanoic acid comprising 2 to 5 carbon atoms or any salts thereof with at least one microorganism capable of carrying out two-carbon chain elongation to produce as an intermediate a linear alkanoic acid comprising 4 to 7 carbon atoms and/or a salt thereof and/or an ester thereof;
(b) extracting the intermediate, its salt and/or ester thereof from (a) using at least one extractant, wherein the extractant comprises at least one alkyl-phosphine oxide and optionally at least one alkane comprising at least 12 carbon atoms; or at least one trialkylamine and at least one alkane comprising at least 12 carbon atoms; or a branched higher alcohol like 2 octyl-dodecanol;
(c) contacting the extracted intermediate and/or ester thereof from (b) and optionally a further linear alkanoic acid comprising 1 to 22 carbon atoms with at least one ketonization catalyst to a linear alkanone comprising 7 to 28 carbon atoms;
(d) contacting the linear alkanone comprising 7 to 28 carbon atoms from step (c) with at least one hydrogenation metal catalyst for catalytic hydrogenation of the linear alkanone comprising 7 to 28 carbon atoms to a corresponding secondary linear alkanol comprising 7 to 28 carbon atoms;
(e) dehydration of the secondary linear alkanol comprising 7 to 28 carbon atoms in the presence of an acidic heterogeneous catalyst to form a corresponding linear alkene comprising 7 to 28 carbon atoms; and
(f) contacting the linear alkene comprising 7 to 28 carbon atoms obtained in step (e) with at least one hydrogenation metal catalyst for catalytic hydrogenation to a corresponding secondary linear alkane comprising 7 to 28 carbon atoms;
or combining step (e) and (f) into one single step using a catalyst or catalyst mixture providing dehydrating and hydrogenating properties, thus hydrogenolysing the secondary linear alkanol comprising 7 to 28 carbon atoms obtained in step (d) directly to the corresponding linear alkane comprising 7 to 28 carbon atoms.

2. The method according to claim 1, wherein the ketonization catalyst of (c) is a metal oxide catalyst or mixtures thereof.

3. The method according to claim 2, wherein the metal oxide catalyst or mixtures thereof is selected from the group consisting of heteropoly acid (H₃PW₁₂O₄₀) catalyst, titanium oxide (TiO₂) catalyst, cerium oxide (CeO₂) catalyst, zinc-chromium (Zn—Cr) mixed oxide catalyst, manganese oxide (MnO₂) catalyst, lanthanum oxide (La₂O₃) catalyst, magnesium oxide (MgO) catalyst, iron oxide (FeO, FeO₂, Fe₂O₃, Fe₃O₄, Fe₄O₅, Fe₅O₆, Fe₅O₇), silicon-aluminium (Si—Al) mixed oxide catalyst and zirconia (ZrO₂) catalyst.

4. The method according to claim 1, wherein the ketonization catalyst in step (c) is manganese oxide (MnO₂) catalyst, or magnesium oxide (MgO) catalyst,.

5. The method according to claim 1, wherein the suitable reaction conditions of step (c) comprises reaction temperatures of 150° C.-350° C.

6. The method according to claim 1, wherein the microorganism in (a) is selected from the group consisting of Clostridium carboxidivorans and Clostridium kluyveri.

7. The method according to claim 1, wherein the alkyl-phosphine oxide is selected from the group consisting of trioctylphosphine oxide, octylphosphine oxide and mixtures thereof and the alkane is selected from the group consisting of pentadecane, hexadecane, heptadecane, octadecane, tetradecane and mixtures thereof.

8. The method according to claim 7, wherein the weight ratio of TOPO to mixture of alkanes is between 1:100 to 1:10.

9. The method according to claim 1, wherein the pH of the aqueous medium in (b) is maintained between 5.5 and 8.

10. The method according to claim 1, wherein the hydrogenation metal catalyst of step (d) is selected from the group consisting of ruthenium (Ru) catalyst, rhenium (Re) catalyst, nickel (Ni) catalyst, iron (Fe), cobalt (Co) and platinum (Pt) catalyst.

11. The method according to claim 1, wherein the acidic heterogeneous catalyst of step (e) is a catalyst comprising an aluminosilicate zeolite.

12. The method according of claim 11, wherein the acidic heterogeneous catalyst of step (e) comprises ZSM-5.

13. The method according to claim 1, wherein the catalyst of step (e) further comprises a transition metal.

14. The method of claim 13, wherein the transition metal is molybdenum.
